(19) United States
(12) Patent Application Publication
(10) Pub. No.: US 2009/0270829 A1
(43) Pub. Date: Oct. 29, 2009

(54) PACKAGED UNDERGARMENT AND CHANGING ACCESSORIES

(76) Inventors: Shaaf Hameed, Grand Rapids, MI (US); Susan E. Perry, Fremont, MI (US)

Correspondence Address: VAN DYKE, GARDNER, LINN & BURKHART, LLP, SUITE 207, 2851 CHARLEVOIX DRIVE, S.E., GRAND RAPIDS, MI, 49546, US

(21) Appl. No.: 12/501,076

(22) Filed: Jul. 10, 2009

Related U.S. Application Data
(63) Continuation-in-part of application No. 11/767,061, filed on Jun. 22, 2007.
(60) Provisional application No. 60/815,767, filed on Jun. 22, 2006.

(51) Int. Cl. A61F 13/15; A61B 19/02

(52) U.S. Cl. 604/385.06; 206/440

(57) ABSTRACT

An undergarment and undergarment changing accessory system includes an undergarment, at least one changing accessory for cleaning or treating an undergarment wearer or caregiver, and a container comprising sheet portions that are partially sealed together with releasable seals along perimeter edge portions that may be readily pulled or peeled apart to expose the undergarment (such as a diaper) and changing accessory contained therein. A fixed or non-releasable seal or fold region may optionally connect the sheet portions after the releasable seals are opened so that the sheet portions form an enlarged sheet that can be used as a sanitary changing surface. A releasable seal established along a divider portion between the sheet portions and separating the undergarment from the changing accessory maintains the undergarment and changing accessory in isolation from one another and from the environment to prevent leaks or contamination prior to opening the releasable seals.

### CROSS REFERENCE TO RELATED APPLICATIONS

The present application is a Continuation-in-Part of U.S. patent application, Ser. No. 11/767,061, filed Jun. 22, 2007, which claims benefit of U.S. provisional application, Ser. No. 60/815,767, filed Jun. 22, 2006, both of which are hereby incorporated herein by reference in their entireties.

### FIELD OF THE INVENTION

The present invention relates generally to diapers or undergarments for infants and/or toddlers and/or adults and, more particularly, to diapers or undergarments and cleaning sheets or “wipes” for infants and/or toddlers and/or adults.

### BACKGROUND OF THE INVENTION

Typically, anytime a parent takes their baby or infant outside of their home, the parent must carry a diaper bag that includes extra diapers and/or cleaning wipes and/or a changing pad for cleaning the baby should the baby have an accident and require changing while on the trip. Often, the wipes are carried in plastic containers that seal the wipes to maintain their moisture. However, such plastic containers often take up a large amount of space in the diaper bag. Further, because the wipes are carried in a separate container, it is often awkward and difficult for the parent to access the wipes while changing the baby. If the parent forgets the diaper bag, or leaves the wipes at home, the parent has no access to the wipes to clean the baby while changing the baby's diaper. Moreover, carrying a diaper bag to accommodate all of the desired changing products and accessories may be cumbersome and burdensome.

Therefore, there is a need in the art for an improved system of providing wipes and a changing surface to assist in cleaning the person when changing the person's diaper or undergarment.

### SUMMARY OF THE PRESENT INVENTION

The present invention provides a packaged diaper or undergarment and diaper-changing accessory so that the undergarment and accessory are substantially sealed within respective pouches or chambers of a package, yet are readily accessible to a user while the packaging materials can be used as a sanitary changing surface, such as for changing a baby's diaper. The undergarment and accessory (such as baby powder, a pre-moistened wipe, a moisturizing lotion, a medicated lotion, a hand sanitizer, or a disposal bag, for example) are readily accessible by a parent or caretaker or user during the changing process. The person changing the diaper or undergarment wearer (such as a baby or child or even adult) thus may readily access the undergarment and changing accessory by peeling apart releasably sealed portions of two sheet portions or films that form the packaging material. A fixedly sealed region of the sheet portions may remain attached or fixed so that the sheet portions remain connected along an edge to form the sanitary changing surface.

According to an aspect of the present invention, an undergarment and undergarment changing accessory system includes an undergarment (such as a diaper or the like), at least one undergarment changing accessory (such as baby powder, a wipe, a moisturizing lotion, a medicated lotion, a hand sanitizer, a disposal bag, gloves, and/or the like), and first and second sheet portions for packaging the undergarment and the changing accessory. The first and second sheet portions cooperatively define at least two chambers that separately receive the undergarment and the changing accessory. The first and second sheet portions are joined to one another along a perimeter, and also along a divider, via releasably sealed seams. The divider separates the chambers and thus separates or isolates the undergarment and the changing accessory while the seams and the divider are sealed. The first and second sheet portions are separable along the releasably sealed seams and at the divider to permit access to the undergarment and the changing accessory while forming a unitary sheet of the first and second sheet portions.

Optionally, the first and second sheet portions may further be joined along a fixedly sealed seam along a perimeter portion or edge of the sealed package or container. The fixedly sealed seam may not be readily separable or releasable, and therefore holds the first and second sheet portions together along edge portions thereof so that the first and second sheet portions may be arranged as a single-layer sheet after the releasably sealed seams and the divider are opened or released or peeled apart.

Optionally, the fixedly sealed seam is an overlapping region of the first and second sheet portions where the sheet portions are fastened or coupled-together, such as, for example, with an adhesive, or via heat-sealing or ultrasonic welding or the like. Optionally, the first and second sheet portions are opposed portions of a single sheet such that the single sheet includes a folded region between the first and second sheet portions when the sheet portions are sealed together. Optionally, the first and second sheet portions form a sanitary changing surface when the releasably sealed seams and the divider are separated or peeled apart. The sheet portions may comprise any suitable material, such as, for example, a polymeric film or sheet.

The releasably sealed seams and the divider may be substantially impervious to liquids and contaminants until being separated or peeled apart, such as to prevent moisture from the changing accessory from soiling the undergarment or from leaking out of the seams, and/or to maintain a sanitary condition inside the chambers defined by the sheet portions.

According to another aspect of the present invention, a method is provided for packaging an undergarment and an undergarment changing accessory. The method includes providing first and second sheet portions in addition to an undergarment and an undergarment changing accessory. The undergarment and the undergarment changing accessory are positioned along the first sheet portion, the first sheet portion is aligned with the second sheet portion in overlapping arrangement, and the first and second sheet portions are releasably sealed together along a perimeter and also across the sheet portions to form a divider between the undergarment and the undergarment changing accessory.

According to yet another aspect of the present invention, a method is provided for carrying and accessing an undergarment and an undergarment changing accessory. The method includes providing first and second sheet portions in addition to an undergarment and an undergarment changing accessory. The first and second sheet portions are releasably sealed to one another along a perimeter and also along a divider to define at least two chambers in which the undergarment and the undergarment changing accessory are separately sealed. The first and second sheet portions are grasped at the perimeter and pulled or peeled away from one another to open the releasably sealed perimeter and the divider. The undergarment and the undergarment changing accessory can then be accessed and removed from the sheet portions.

Optionally, the first and second sheet portions are joined in part along a fixedly sealed portion so that when the sheet portions are pulled or peeled away from one another, the sheet portions remain attached to one another at the fixedly sealed portion. The sheet portions may then be laid on a support surface, such as to provide a sanitary surface on which to change a baby using the undergarment or diaper and the changing accessory.

Therefore, the present invention provides a packaged diaper or undergarment and diaper-changing accessory that permits storage and carrying of the diaper and changing accessory that are substantially sealed within separate chambers of a package that is readily peeled apart to access the diaper and changing accessory. The package is made up of sheets or films or sheet members, such as plastic or polymeric films, which may be joined or sealed together to form seams, such as releasable seams and at least one fixed seam. The seams permit the package to be opened while retaining the sheet portions together along an edge portion so that the sheet portions may be used as a sanitary changing surface. Thus, the present invention substantially enhances the diaper changing process and/or undergarment wearer cleaning or treating process, since the changer/cleaner/treater can carry everything needed for diaper changing in a convenient package, and need not seek out a sanitary surface or sanitize a non-sanitary surface. Rather, and in accordance with the present invention, a user or changer or parent or caretaker may now readily transport a diaper and diaper-changing accessories in a self-contained kit that includes a sanitary and disposable changing surface.

These and other objects, advantages, purposes and features of the present invention will become apparent upon review of the following specification in conjunction with the drawings.

### BRIEF DESCRIPTION OF THE DRAWINGS

FIG. 1 is a perspective view of an undergarment and wipe system in accordance with the present invention;

FIG. 2 is another perspective view of an undergarment and wipe system of the present invention, showing the outer containing element partially removed from the surface of the undergarment and a wipe being removed from the wipe receiving cavity;

FIG. 3 is a sectional view taken along the line III-III in FIG. 1;

FIG. 4 is another sectional view similar to FIG. 3, showing another wipe containing element in accordance with the present invention;

FIG. 5 is a sectional view of another wipe containing element in accordance with the present invention;

FIG. 6 is a perspective view of another undergarment and wipe system of the present invention, with a cover film that substantially envelops the entire undergarment, shown with the cover film opened to expose and access a wipe contained therein;

FIG. 7 is a sectional view of the undergarment and wipe system of FIG. 6, shown with the cover film wrapped around the undergarment;

FIG. 8 is a sectional view of a packaged undergarment and changing accessory system in accordance with the present invention;

FIG. 9 is a sectional view of the packaged undergarment and changing accessory system of FIG. 8, shown in an unfolded configuration;

FIG. 10 is a top plan view of the packaged undergarment and changing accessory system of FIG. 9; and

FIG. 11 is a top plan view of the packaged undergarment and changing accessory system of FIG. 10, shown in an opened configuration.

### DETAILED DESCRIPTION OF THE PREFERRED EMBODIMENTS

Referring now to the drawings and the illustrative embodiments depicted therein, an undergarment or diaper and wipe system 10 includes an undergarment or diaper 12, such as a disposable diaper or the like, a wipe containing element 14 and at least one cleaning wipe 16 contained at or within the wipe containing element 14 and at a surface 12a of undergarment 12 (FIGS. 1-3). Wipe containing element 14 is attached or adhered to surface 12a of undergarment 12 to contain and substantially seal the wipe or wipes 16 within a wipe receiving cavity 18 at the surface 12a of undergarment 12, such as at an exterior surface at the rear portion of the undergarment or diaper. Accordingly, the wipes are readily accessible by a user or changer, such as a parent or babysitter or caretaker or the like, during the process of changing a wearer (such as a baby or child or adult—where the diaper and/or wipes may be provided in a smaller child size and a larger adult size to accommodate the different sized wearer's and associated accidents/cleanups, or may be provided with different cleaning or treating agents as discussed below), since the wipes are attached to or contained at the undergarment or diaper itself and not in a separate plastic container in a diaper bag. The wipes and wipe containing element are thus integrated with the undergarment or diaper itself and thus provide for enhanced access of the wipes and cleaning of the wearer, as discussed below. Optionally, the wipe containing element may be removably attached to the surface of the undergarment or diaper and may be readily removed therefrom to provide a separately portable, self-contained, sealed wipe containing element or package that is separate from the undergarment, as also discussed below.

Undergarment or diaper 12 may comprise any known type of diaper or protective undergarment, such as a disposable diaper having a water-resistant or plastic or polymeric type exterior skin or surface 12a. Such diapers are well-known in the art, such that a detailed discussion of the diaper itself need not be included herein. Although shown and described as a disposable diaper, it is envisioned that aspects of the present invention are equally suitable for application to non-disposable diapers, while remaining within the spirit and scope of the present invention. It is also envisioned that the present invention is highly suitable for applications on and with adult protective or absorbent undergarments and the like, and is not limited to applications on baby diapers and the like.

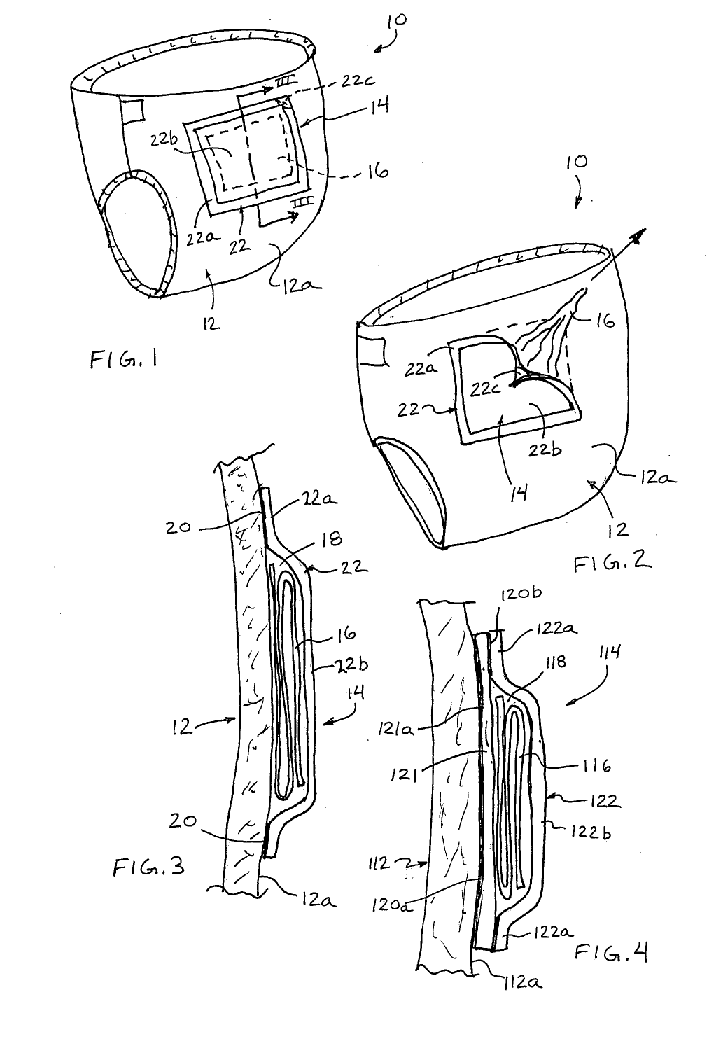

Wipe containing element 14 is attachable to surface 12a of diaper 12 via an adhesive layer 20 (FIG. 3), such as a pressure sensitive adhesive or any other suitable adhesive or tape or other attaching means. As shown in FIG. 3, wipe containing element 14 may comprise an outer cover panel or element or layer 22, which is adhered or attached to surface 12a of diaper 12 about its perimeter region 22a, whereby a central region 22b of cover panel 22 functions to define wipe receiving cavity 18 at surface 12a of diaper 12. Preferably, and as can be seen in FIG. 1, a corner or portion 22c of cover panel 22 may not be attached to or adhered to surface 12a of diaper 12, so that corner portion or perimeter portion 22c provides a grasping portion for a user to grasp and pull so as to readily peel away the cover panel 22 from surface 12a of diaper 12 and thus to readily access the wipe or wipes 16 contained in wipe receiving cavity 18.

Preferably, the attaching means or adhesive 20 used to attach or adhere perimeter portion 22a of cover panel 22 to surface 12a of diaper 12 comprises an adhesive that is sufficient to substantially seal the cover panel 22 to the surface 12a so as to substantially seal and provide a substantial airtight and watertight seal around cavity 18 so as to limit or substantially preclude drying out of the wipes 16 contained therein. However, the selected adhesive or attaching means is also preferably selected so that perimeter portion 22a of cover panel 22 may be readily removed or peeled away from surface 12a of diaper 12, preferably without damaging the surface of the diaper.

Optionally a portion of the perimeter of the cover panel 22 may be removably adhered or attached (such as via a pressure sensitive adhesive or the like) to the diaper surface, while a corner or perimeter portion of the cover panel 22 may be substantially permanently attached or adhered or affixed to the diaper surface, so that the cover panel is not readily entirely removable from the diaper. Such a configuration keeps the cover panel at the diaper so it does not blow away or get lost and thus not get disposed of properly with the diaper, and also allows for easier resealing of the wipe containing element by keeping the cover panel at least partially attached to the diaper surface.

Accordingly, one or more cleaning wipes 16 may be stored within the cavity 18 defined between the cover panel 22 and the surface 12a of diaper 12, and may be readily accessed and removed therefrom by a user or parent or diaper changer. As can be seen in FIG. 3, the wipe or wipes may be folded within the wipe receiving cavity 18 so as to minimize the space taken up by the wipes, such that the addition of the wipes and wipe containing element at the exterior surface of the diaper is substantially unnoticeable by the wearer of the diaper, in order to maintain the diaper wearer's comfort while wearing the diaper. The number of wipes contained within the wipe receiving cavity may be selected to provide enough wipes to sufficiently clean a baby or diaper wearer, while limiting the thickness of the wipes within the cavity so as to limit the additional material at the exterior of the diaper, in order to maintain the wearer's comfort when wearing the diaper.

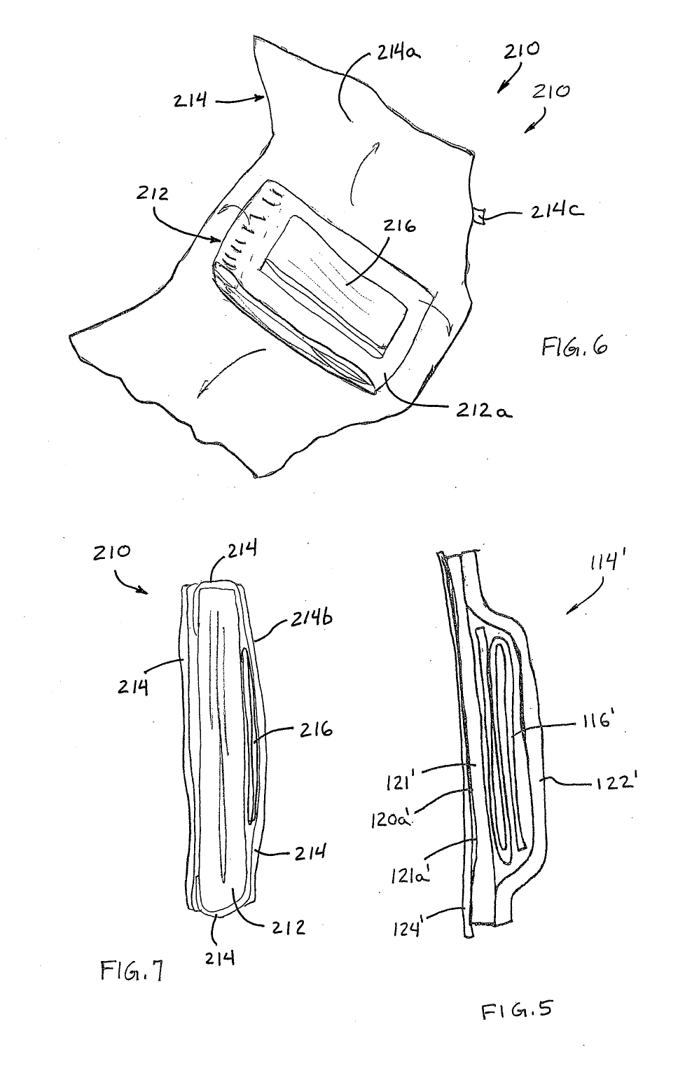

Optionally, and with reference to FIG. 4, a wipe containing element 114 may be attached to an exterior surface 12a of an undergarment or diaper 112 to contain one or more wipes 116 at the undergarment or diaper 112, such as in a similar manner as described above. As can be seen in FIG. 4, wipe containing element 114 includes an inner attaching panel or element or sheet portion 121 that is attachable to surface 112a of diaper 112, such as via an adhesive or tape or the like 120a, and an outer cover panel or element 122. Outer cover panel or element or sheet portion 122 is attached to or adhered to attaching element 121 (such as via an adhesive or tape or other suitable attaching or adhering means 120b) around the perimeter portion 122a of cover element 122 so as to define a wipe receiving cavity 118 between a generally central portion 122b of cover element 122 and attaching element 121.

Thus, the wipes 116 are contained within the wipe containing element 114 itself, whereby the wipe containing element may be separately attached to the exterior surface 112a of the diaper 112. Similar to wipe containing element 14, described above, wipe containing 114 may include a grasping portion at the perimeter portion 122a of cover element 122 to assist a user in grasping and peeling the cover element 122 at least partially away from the attaching element 121, so as to access the wipes 116 and remove the wipes 116 from cavity 118. Although shown as two separate sheets or elements, it is envisioned that a single sheet may be folded over on itself to form the attaching element and the cover element, with the opposed sheet portions being selectively sealed or adhered together about their opposed perimeter regions to form the wipe receiving cavity, without affecting the scope of the present invention.

Optionally a portion of the perimeter of the cover panel 122 may be removably adhered or attached (such as via a pressure sensitive adhesive or the like) to the attaching element, while a corner or other perimeter portion of the cover panel 122 may be substantially permanently attached or adhered or affixed to the attaching element, so that the cover panel is not readily entirely removable from the attaching element. Such a configuration keeps the cover panel at the attaching element and/or undergarment/diaper so it does not blow away or get lost and thus not get disposed of properly with the wipe container or pouch or diaper, and also allows for easier resealing of the wipe containing element by keeping the cover panel at least partially attached to the attaching element.

Optionally, the adhesive used to secure the cover panel to the outer surface of the attaching element may be a reusable adhesive, so that the cover element may be partially peeled away and a wipe may be removed from the wipe receiving cavity, and the cover element may then be resealed to the surface of the attaching element. Thus, a user may access a cleaning wipe for cleaning up spills or the like, while one or more wipes may still be present within the wipe receiving cavity 118 for use later when the diaper needs changing.

Optionally, the wipe containing element 114 may be removably attached to the surface of the diaper (such as via a pressure sensitive adhesive at the attaching element 121) so that the user may remove the wipe containing element 114 from the surface of the diaper and carry the wipe containing element as a separately portable, self contained, sealed wipe containing element or package or flexible pouch, separate from the diaper. The attaching element 121 may have a pressure sensitive adhesive for removably attaching the attaching element and wipe containing element to the surface of the diaper, or the attaching element may utilize other adhesives or the like for the removably attachment.

Thus, wipe containing element may be attached to the surface of the undergarment or diaper during manufacturing of the undergarments or diapers, whereby the undergarment/diaper and wipe system may be sold as a unitary item. However, because the wipes may be contained within the wipe containing element itself, it is envisioned that the wipe containing element may be provided or sold as separate item or unit, whereby a user may attach the wipe containing element and the wipes contained therein to a surface of an undergarment or diaper or to another surface if desired. For example, and with reference to FIG. 5, it is envisioned that an attaching surface 121a′ of an attaching element or sheet portion 121′ of a wipe containing element 114′ may have an adhesive layer 120a′ established thereon, such as a pressure sensitive adhesive or such as any other suitable adhesive or tape, and may be provided initially with a peel away cover layer 124′ over the adhesive. A user may then peel away the cover layer to expose the adhesive and then may adhesively attach or secure the wipe containing element to the surface of the undergarment or diaper.

Thus, the wipe containing element 114′ may be sold or provided as a separate package or pouch of wipes, separate from the diapers, whereby a user may carry the separate pouch of wipes separately from the diaper or may attach the separate pouch of wipes to the diaper (such as after separately purchasing the diapers and the separate pouches). In such an application, the pouch may be provided with the removable cover film 124′ that is removably at and covering the attaching portion of the attaching element 121′ to cover and seal against any adhesive 120a′ at the attaching element. Thus, if a user desires to attach the wipe containing element 114′ to a surface of a diaper, the user may remove the cover film 124′ from the attaching element 121′ (so as to expose an adhesive at the attaching element 121′) and may press the wipe containing element 114′ to the diaper to adhesively attach the attaching element of the wipe containing element to the diaper surface. The cover film 124′ covers and at least partially seals against the adhesive layer at the attaching element to limit or substantially preclude unintentional attachment of the wipe containing element to other items until it is ready for application or attachment to the diaper surface (or other targeted surface or object). The outer cover panel 122′ is at least partially removably attached to the attaching element 121′ so as to define the cavity therebetween for containing the wipe or wipes or towels 116′, such as in a similar manner as described above.

Optionally, and with reference to FIGS. 6 and 7, an undergarment or diaper and wipe system 210 includes an undergarment or diaper 212, such as a disposable diaper or the like, a wipe containing element or cover or film 214 and at least one cleaning wipe 216 contained at or within the wipe containing element 214 and at a surface 212a of undergarment 212. Cover film 214 may comprise a thin plastic sheet or film that is attached or adhered to surface 212a of undergarment 212 to contain and substantially seal the wipe or wipes 216 at the surface 212a of undergarment 212. The wipe or wipes 216 thus may be sealed at the outer surface of the diaper (such as at the substantially water tight or water impervious outer layer of a disposable diaper), with the cover film substantially encasing or enveloping and sealing the wipe or wipes thereat, so that the moistened or saturated wipes maintain their moistness for use.

As can be seen with reference to FIG. 6, the cover film 214 is sized so as to be wrapped at least partially around the diaper 212. The inner surface 214a of cover film 214 may be a light tack surface or partially adhesive surface so that the cover film may be wrapped around the diaper and substantially sealed to the diaper or to the outer surface 214b of cover film 214 (where the cover film may overlap itself when wrapped around the diaper), yet may be readily peeled away from the diaper or cover film to access the wipe 216 contained or sealed therein. The cover film 214 thus may substantially envelop the diaper, and functions to seal at least partially around the wipe 216 (which may be contained within a pocket formed by the cover film or may be contained between the cover film and the outer surface of the diaper) to contain and seal the wipe at the diaper.

Thus, when the user or caretaker is to change the diaper wearer, the user takes a new diaper (as wrapped and sealed by the cover film 214) and unwraps the new diaper and accesses the wipe contained thereat (whereby the wipe or wipes may be used to clean the diaper wearer before changing the diaper). Optionally, it is envisioned that, due to the size of the cover film, the cover film, when opened or removed or partially removed from the new diaper, can be used as a mat for the baby to be placed on during the changing process. For example, the caretaker may remove the cover film from the new diaper and may flip the cover film over so that the tacky side is down (against the support surface or table or floor where the baby will be changed), so that the cover film may remain in place at the support surface. The baby may then be placed on the cover film during the changing process so that the baby does not have to come in contact with the support surface or table or floor, which may be unsanitary. After the changing is completed, the cover film may be used to wrap up the dirty diaper and substantially seal the dirty diaper in a compact, sealed manner, so that the caretaker may later properly dispose of the dirty diaper.

Optionally, the cover film or sheet and/or diaper contained therein may be color coded or marked so that a user (such as a nurse at a hospital or health care facility or a nurse or caretaker at a nursing home or the like) may readily discern the size or type of diaper/undergarment contained in the package, so that the user is able to readily use the appropriate size or type for the particular patient. For example, the cover film 214 may include a color coded tab 214c or may otherwise have a coloring (for example, the cover film may be dyed different colors for different applications) to facilitate recognition of the different sizes or types of diapers or undergarments.

Optionally, some of the cover films and undergarments may be provided with other items other than wipes (or in addition to wipes, such as by having a wipe or wipes at one side of the diaper and another item or items at the other side of the diaper, with both or all items substantially enveloped and/or sealed within the cover film), such as, for example, medical gloves or bandages or tape or other medicine or devices or the like to assist in treating or caring for the skin condition or health condition of the wearer. Optionally, different codings or indicia at the cover film or undergarment could also indicate different items that may be contained or wrapped within the cover film. For example, a cover film may be marked so that a user may readily recognize that the cover film contains gloves or the like therein (either alone or in addition to the wipes, such as at an opposite side of the undergarment from the wipes), such as for medical applications where the nurse or caretaker should wear gloves during the treatment or cleaning process (such as when there is an open wound or when hazardous chemicals are used or the like). Other variations may be provided while remaining within the spirit and scope of the present invention.

The present invention thus provides a container or pouch or packet or cover for containing and/or sealing one or more wipes or cleaners, which may be treated or soaked in a cleaning agent or medicinal agent or ointment or the like, and which may be readily applied to a diaper or garment so that they are readily accessible to the wearer of the garment or the caretaker of the wearer. For example, an elderly person may have one or more packets attached to their undergarment (or one or more items sealed within the cover film) so that the wipes are readily accessible, such as for cleaning the person or treating a skin condition (such as a cut or abrasion or burn or rash or allergic reaction or the like) of the person or cleaning of the person (such as if the person has a bladder control problem or the like) via the appropriately treated wipe or wipes.

Optionally, it is envisioned that the containers or packets or pouches of the present invention may be provided with a variety of different contents or wipes, such as wipes or cloths soaked or treated with different chemicals or medicines or ointments or the like. For example, one packet of wipes may contain wipes treated with a cleaning agent, while another packet of wipes may contain wipes treated with an allergy medicine, and another packet of wipes may contain wipes treated with a burn medication, and another packet of wipes may contain wipes treated with a rash medication, and/or the like. Thus, the appropriate packet may be readily selected and applied to the wearer's undergarment so that it is readily accessible by the wearer or wearer's caretaker when needed (or optionally, an appropriate undergarment and cover film may be selected depending the condition or needs of the patient or wearer). Optionally, some of the packets may be provided with other items other than wipes, such as, for example, bandages or tape or other medicine or devices or the like to assist in treating or caring for the skin condition or health condition of the user.

Optionally, such packets or containers may be provided as color-coded packets or containers so that the caretaker or user may readily identify and select the appropriate or desired packet and wipes/items contained therein. For example, an allergy treatment packet (containing wipes or cloths treated with an allergy medicine, such as may be used or applied as a topical medicine to treat a skin condition due to allergies or the like) may be colored red, while a rash treatment packet (containing wipes or cloths treated with a rash ointment or medicine, such as may be used or applied as a topical ointment or medicine to treat a skin condition or rash) may be colored blue, and a burn treatment packet (containing wipes or cloths treated with a burn ointment or medicine, such as may be used or applied as a topical ointment or medicine to treat a burn area of a patient) may be colored yellow, and a cut or abrasion treatment packet (containing wipes or cloths treated with an antiseptic or anti-bacteria or anti-infection medicine or ointment and/or bandages or the like) may be colored green (or any other color as selected or desired). Different shades of some of the colors may be used to indicate different types of treating agents within a common category of ailments or conditions.

Thus, a caretaker or a doctor or nurse at a health facility may readily identify and select the appropriate packet for a particular patient and may apply the packet to the patients garments or undergarments as described above (or may select the appropriate cover film and undergarment unit), such that the appropriate or desired medication or ointment or wipe is readily available to the patient or the patient's caretaker when needed. Optionally, although described as color-coded packets or containers, the packets or containers or cover films may be otherwise coded or marked (such as via alphanumeric characters or icons or indicia or the like) to indicate which type of wipes (or other items) are contained therein, while remaining within the spirit and scope of the present invention. Thus, the undergarment wearer may be readily treated (such as via a cleaning wipe or medicinal wipe or the like) when such treatment is desired or appropriate (such as after an accident or when it is time for a regularly scheduled cleaning or medicinal application or the like), without having to look for the appropriate wipes or cloths.

The present invention thus provides an enhanced means for carrying cleaning wipes or otherwise treated wipes or cloths or other items for cleaning or treating a garment wearer. For example, when it is time to change a diaper of a child or baby, the person changing the diaper may readily grasp a grasping portion of a peel away cover element and may partially peel the cover element away from the diaper so as to access the wipe receiving cavity and the wipes contained therein. The user may then readily grasp and pull one or more wipes (or other items) from the cavity for use in cleaning or treating the diaper wearer. After the wearer is cleaned and/or treated and/or changed, the dirty diaper, wipes and wipe containing element may all be discarded together. The new clean diaper then on the baby or diaper wearer would also include its own self-contained wipes for cleaning the wearer during the next changing process. Similarly, for an adult user of the system of the present invention, the appropriate wipe or cloth or treating agent may be selected and attached to the user's garments so that it is likewise readily accessible when needed or desired.

Therefore, the present invention provides a unitary undergarment/diaper and wipe system so that a parent or babysitter or caretaker or the like will almost always have cleaning and/or treating wipes readily available for use in cleaning or treating the garment wearer, such as, for example, when changing the wearer's diaper. The present invention thus avoids the frustration and difficulties encountered when trying to hold a baby with one hand and access wipes in a separate container in a diaper bag or the like, as is often required during the changing process of an angry or uncomfortable child. The present invention also avoids the chance of the parent or babysitter or caretaker forgetting to put the separate wipe container in a diaper bag or case or the like before heading out on an errand or the like, since as long as the diapers are available, the wipes are also available at the diaper. Optionally, the wipe containers of the present invention may be detached from the diaper and carried as separate sealed packages separate from the diaper, if desired. The present invention also substantially limits or precludes or avoids the likelihood of a patient being without the appropriate treatment since the treating agent or agents are provided at or attached to or integrated with the patient's garments or undergarments.

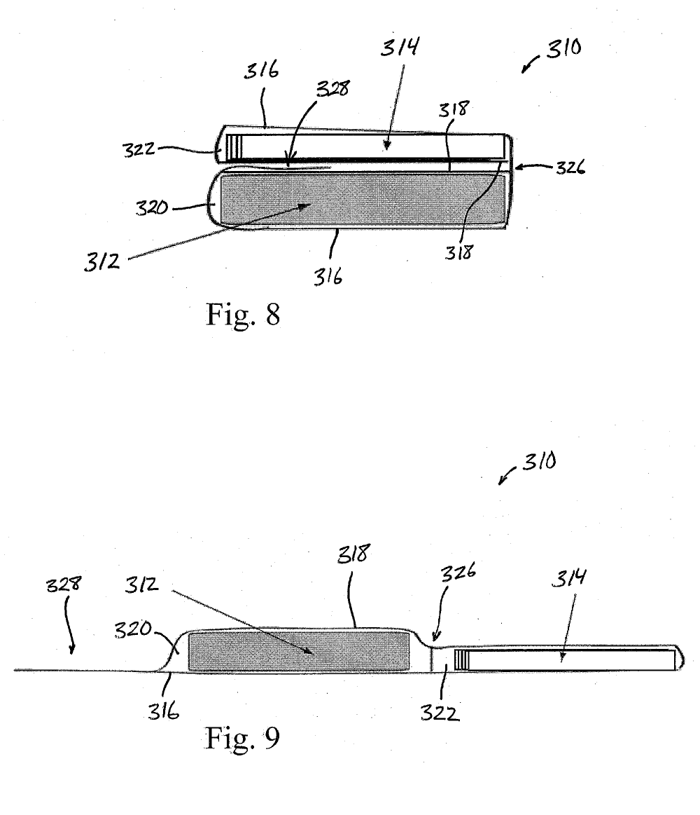

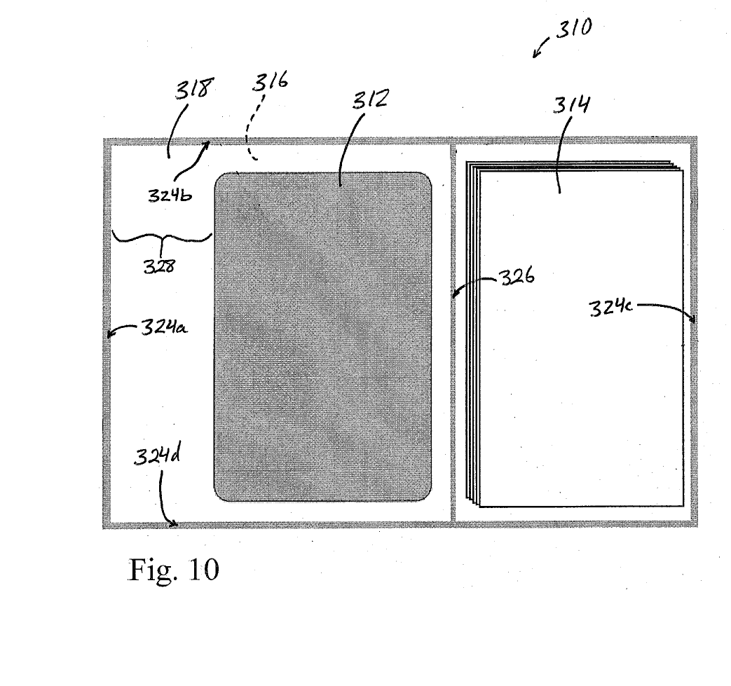

Optionally, and with reference to FIGS. 8-11, a packaged undergarment and wipe system 310 includes an undergarment or diaper 312, such as a disposable diaper or the like, a diaper-changing accessory 314, and first and second sheet portions 316, 318 cooperating to contain the diaper and changing accessory in a substantially sealed manner in first and second pouches or chambers 320, 322, respectively (FIGS. 8 and 9). Sheet portions 316, 318 may comprise thin plastic sheets or films that are sealable to one another in varying degrees to form chambers 320, 322. In the illustrated embodiment, first sheet portion 316 is releasably sealed to second sheet portion 318 along three perimeter edge portions 324a-c and along a divider portion 326 that extends between and separates the pouches or chambers 320, 322. First sheet portion 316 is fixedly sealed to second sheet portion 318 along a fourth perimeter edge portion 324d (FIG. 10), as will be described in greater detail below.

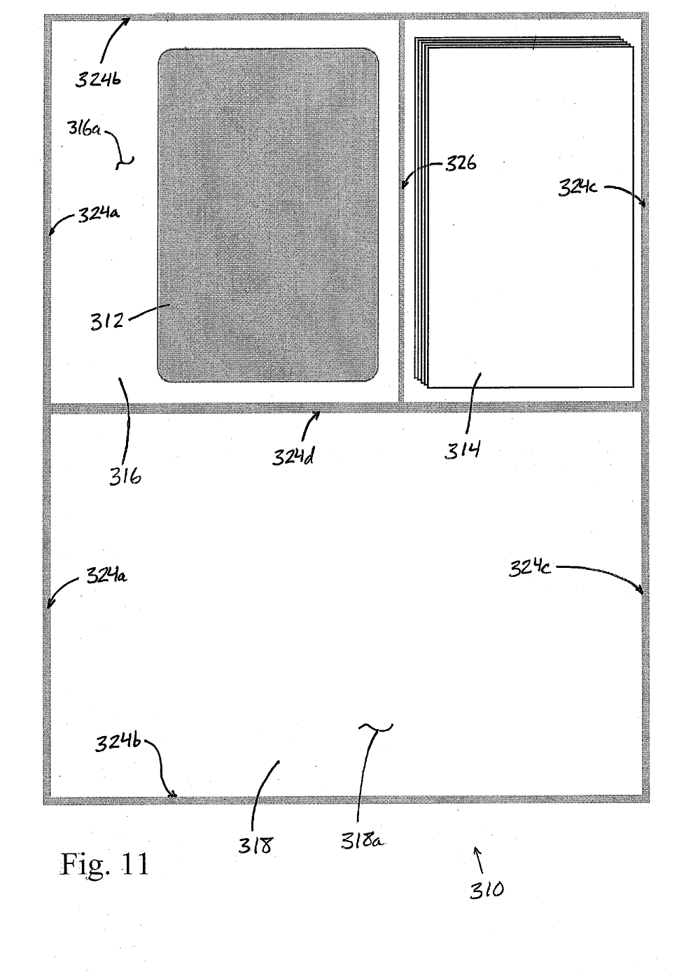

The sheets or sheet portions may comprise separate sheets or films or panels overlaid and sealed together around their perimeter regions, or may comprise a single sheet or film folded over on itself and sealed together about its perimeter regions (with the folded joint or portion of the single sheet functioning as the fixedly sealed seam or perimeter edge), while remaining within the spirit and scope of the present invention. The sheets or sheet or sheet portions may comprise any suitable material, such as metallic foil, a flexible polymeric or plastic sheet material, or any other material that may be suitable for containing either dry or moist materials or articles such as, for example, diaper 312 (which may be packaged with other dry articles such as gloves, sealed packages of lotion or medicaments, and the like) and diaper-changing accessory 314 (which may comprise, for example, baby powder, a pre-moistened wipe, a moisturizing lotion, a medicated lotion, a hand sanitizer, a disposal bag, or the like). Optionally, inner surfaces 316a, 316b (FIG. 11) of the sheet portions 316, 318 may be maintained in a sanitary condition, and may be sanitized or sterilized during manufacturing or assembly of system 310.

Releasable seals along perimeter edge portions 324a-c and divider portion 326, which join first sheet portion 316 to second sheet portion 318, serve to maintain chambers 320, 322 in a sealed condition (i.e. substantially preventing both leakage of the chamber contents and contamination of the chamber contents from the environment, thus keeping the contents of chamber 320 sanitary and dry, and keeping the contents of chamber 322 sanitary and dry or wet, depending on the contents), while being readily openable, preferably without tearing. For example, sheet portions 316, 318 may be releasably joined to one another along perimeter edge portions 324a-c and divider portion 326 via ultrasonic welding, heat sealing, an adhesive, or the like.

Regardless of the method used to form releasable seals, the releasable seals are readily openable by applying deliberate pulling or peeling forces upon the two sheet portions to separate them from one another at perimeter edge portions 324a-c and divider portion 326. Although shown and described as having a single divider portion comprising a releasable seal, it will be appreciated that substantially any number of chambers may be formed with substantially any number of dividers or divider portions, depending on the particular application of the system. Optionally, one or more of the perimeter edge portions 324a-c may include a non-sealed corner or edge portion to initiate opening the releasable seals via the application of pulling or peeling forces (by allowing a user to readily grasp the unsealed edge portions and initiate the peeling process).

Optionally, the releasable seals along perimeter edge portions 324a-c and divider portion 326 may be re-sealable, such as to permit reuse of first and second sheet portions 316, 318. For example, re-sealable seals such as those commonly used on plastic bags (e.g. ZIPLOC® brand bags) may be used without departing from the spirit and scope of the present invention. Re-sealable seals may be pulled or peeled apart in a similar manner as described above, but also may be re-sealed, such as for providing a sanitary disposal package for a soiled undergarment and used changing accessories, or for permitting a fresh undergarment and/or changing accessories to be sealed between previously-used sheet portions.

Optionally, the inner surfaces 316a, 316b of sheet portions 316, 318 may comprise a light tack surface or partially adhesive surface so that the sheet portions may be selectively sealed together by applying pressure along perimeter edge portions 324a-c and divider 326, yet may be readily peeled apart from one another to access the diaper 312 and changing accessory 314 contained or sealed therein. The sheet portions 316, 318 thus may substantially envelop the diaper and changing accessory, and function to seal around the diaper and changing accessory to prevent leaks and/or contamination. Optionally, it is envisioned that, due to the size of the sheet portions, the sheet portions can be used as a mat for a baby to be placed on during the changing process. For example, the caretaker may peel apart the sheet portions and flip the sheet portions over so that the tacky side is down (against the support surface or table or floor where the baby will be changed), so that the sheet portions may remain in place at the support surface. After the changing is completed, the tacky sheet portions may be used to wrap up the dirty diaper and changing accessories, and substantially seal them in a compact, sealed manner, so that the caretaker may later properly dispose of the soiled articles.

In the illustrated embodiment, perimeter edge portion 324d is a region in which first and second sheet portions 316, 318 are sealed together in a manner that substantially prevents them from separating from one another in that region. Perimeter edge portion 324d comprises a fixed or non-releasable seal that may be formed using a similar method as used for forming non-releasable seals, such as ultrasonic welding, heat sealing, adhesive bonding, or the like. However, it will be appreciated that the method used for forming the fixed or non-releasable seal along perimeter edge portion 324d creates a higher-strength bond than that of the releasable seals described above. For example, this may be accomplished with a higher-intensity or longer-duration ultrasonic weld or heat seal, or with a stronger adhesive than is used in forming the releasable seals elsewhere on the sheet portions. Optionally, first and second sheet portions 316, 318 may be unitarily formed of a single sheet or film, where the sheet portions comprise opposing sides of the single sheet. The opposing sides are folded over one another so that a fixed or non-releasable seal is not needed for joining the first and second sheet portions 316, 318 together.

Optionally, first and/or second sheet portions 316, 318 may be somewhat oversized (beyond what is necessary for containing the diaper and changing accessories), such as to form a suitably-sized sanitary changing surface or barrier that may be laid flat on a changing surface after the sheet portions are partially peeled or separated from one another along three sides, leaving a fourth side unpeeled. For example, and with reference to FIGS. 8-10, an excess-width region 328 of first and second sheet portions may be provided between perimeter edge portion 324a and chamber 320. Excess-width region 328 is either substantially unsealed or releasably sealed depending on the amount of space desired in chamber 320 for diaper 312. The excess-width region 328 increases the surface area available for sanitary changing of a baby, for example, as in FIG. 11 and described below. The sheets may be sealed at the perimeter edge 324a or across the excess-width region 328 or at or adjacent to chamber 320 (where the sheets at the excess-width region may not be sealed together to facilitate grasping and peeling of the sheets by the user).

Accordingly, one or more undergarments or diapers 312 may be packaged together with one or more diaper-changing accessories 314 in separate chambers 320, 322 and maintained in a sanitary condition during storage and carrying of the system 310. Optionally, the sheet portions 316, 318 may be folded over one another, such as at divider 326, and the excess-width region 328 tucked between the portions of second sheet portion 318 that are folded over into close proximity to one another to provide a compact package (FIG. 8). Optionally, a connector, such as a button snap, hook and loop fastener, magnets, adhesive tape, or the like, may be used to secure system 310 in a folded or compact configuration.

To open and access the contents of the package, sheet portions 316, 318 are unfolded (FIGS. 9 and 10) so that perimeter edge regions 324a-c are accessible for opening the releasable seals established therealong. Second sheet portion 318 is peeled away from first sheet portion 316 such as by grasping the sheet portions in the region of one of chambers 320, 322, or along one of perimeter edge regions 324a-c and pulling or peeling them apart. The fixedly sealed perimeter edge portion 324d remains sealed or joined so that sheet portions 316, 318 remain attached to one another as the contents of chambers 320, 322 are exposed.

When the releasable seals are unsealed or opened (FIG. 11), diaper 312 and changing accessory or accessories 314 are exposed and immediately accessible and removable from the package or system, without need for opening additional wraps or packaging. First sheet portion 316 and second sheet portion 318 remain joined to one another along an overlapping region along the fixed or non-releasable seal at perimeter edge portion 324d (or along an unfolded fold line if the sheet portions are formed from a continuous sheet) to form a sanitary changing surface at inner surfaces 316a, 318a, such as may be used between a baby and an unsanitary support surface during the diaper-changing process, or to prevent a changing support from becoming soiled.

Undergarment or diaper 312 and changing accessories 314 may be accessed or removed from first sheet portion 316a and, in the case of a small child or infant being changed, the child may be placed atop the exposed inner surfaces 316a, 318a of sheet portions 316, 318 for changing with the diaper and/or for treatment with the one or more changing accessories. After the changing process is complete, soiled diapers and changing accessories may be placed on the sheet portions and bundled for disposal, or the soiled materials may be placed in a disposable bag provided for that purpose and later discarded of at the user's convenience.

Thus, an undergarment or diaper may be packaged together with one or more changing accessories, such as moist wipes, baby powder, sanitizing or medicated lotions, and the like, between sheet portions that both form a sealed and sanitary package or container that is easily transported, such as in a purse or a pocket, and that provide a sanitary changing surface on which a young child or patient may have its diaper changed without concern for exposing the child to unsanitary conditions, or for exposing a changing support to soiled articles resulting from the changing process. The sheet portions are readily peeled or pulled apart for access to the diaper and changing accessories, yet the releasable seals along perimeter edge portions and a divider are substantially moisture-proof so that both wet articles (such as wipes) and dry articles (such as diapers) may be packaged together without risk of contamination of the articles or leaking of fluids. The selectively sealed packages contain any other types of changing or cleaning or treating wipes or cloths, such as medicated cloths as discussed above, and/or other medical or medicated items, and/or may be color coded to assist in identification of the type of cloth contained therein, as also discussed above.

It will be appreciated that substantially any size of undergarment may be packaged and used as described above, including adult-size diapers. The packaged undergarment and changing accessories permit these articles to be carried discreetly, such as in a pocket or a purse, and without need for a separate diaper bag, for example. Thus, an adult may discreetly carry a diaper with changing accessories on their person or in a purse, use the articles privately, such as in a bathroom stall, and dispose of the soiled articles in the garbage without drawing undesired attention, such as may occur by carrying a diaper bag.

Changes and modifications to the specifically described embodiments may be carried out without departing from the principles of the present invention, which is intended to be limited only by the scope of the appended claims, as interpreted according to the principles of patent law.

1. An undergarment and undergarment changing accessory system, said system comprising:
an undergarment;
at least one undergarment changing accessory;
first and second sheet portions comprising first and second perimeter portions with releasably sealed seams along said first perimeter portion to define at least two chambers between said sheet portions for containing said undergarment and said at least one undergarment changing accessory;
a divider between said at least two chambers, said divider adapted to initially separate said undergarment from said at least one undergarment changing accessory; and
wherein said first and second sheet portions are separable at said releasably sealed seams and at said divider to permit access to said undergarment and said at least one undergarment changing accessory, said first and second sheet portions forming a unitary sheet when separated at said releasably sealed seams, with said first and second sheet portions joined at said second perimeter portion.

2. The system of claim 1, further comprising a fixedly sealed seam along said second perimeter portion at least partially defining at least one of said at least two chambers, said fixedly sealed seam adapted to maintain said first and second sheet portions in coupled arrangement so that said first and second sheet portions form a single-layer sheet when said releasably sealed seams and said divider are separated.

3. The system of claim 2, wherein said fixedly sealed seam comprises an overlapping region of said first and second sheet portions.

4. The system of claim 1, wherein said first and second sheet portions comprise opposed portions of a continuous sheet, and wherein said continuous sheet comprises a folded region between said first and second sheet portions when said sheet portions are sealed together.

5. The system of claim 2, wherein said first and second sheet portions provide a sanitary changing surface when said sheet portions are separated.

6. The system of claim 1, wherein said first and second sheet portions each comprise a polymeric film.

7. The system of claim 1, wherein said undergarment comprises a disposable diaper.

8. The system of claim 7, wherein said at least one undergarment changing accessory comprises at least one selected from baby powder, a pre-moistened wipe, a moisturizing lotion, a medicated lotion, a hand sanitizer, and a disposal bag.

9. The system of claim 1, wherein said releasably sealed seams and said divider are substantially impervious to liquids and contaminants until said first and second sheet portions are separated.

10. The system of claim 9, wherein said releasably sealed seams and said divider comprise at least one selected from (a) an adhesive between said first and second sheet portions, (b) ultrasonically welded portions of said first and second sheet portions, and (c) heat sealed portions of said first and second sheet portions.

11. A method of packaging an undergarment and an undergarment changing accessory, said method comprising:
providing an undergarment, an undergarment changing accessory, and first and second sheet portions;
positioning said undergarment and said undergarment changing accessory along said first sheet portion;
aligning said first sheet portion with said second sheet portion in overlapping arrangement;
releasably sealing said first and second sheet portions together along a first perimeter portion and at least partially around said undergarment and said at least one undergarment changing accessory; and
releasably sealing said first and second sheet portions together between said undergarment and said at least one undergarment changing accessory.

12. The method of claim 11, further comprising fixedly sealing said first and second sheet portions together along a second perimeter portion.

13. The method of claim 11, wherein said aligning said first and second sheet portions comprises folding said first sheet portion over said second sheet portion to form a second perimeter portion of said first and second sheet portions.

14. The method of claim 11, wherein said releasably sealing said first and second sheet portions together comprises at least one selected from (a) applying an adhesive between said first and second sheet portions, (b) ultrasonically welding portions of said first and second sheet portions, and (c) heat sealing portions of said first and second sheet portions.

15. The method of claim 11, wherein said undergarment comprises a disposable diaper.

16. The method of claim 15, wherein said undergarment changing accessory comprises at least one selected from baby powder, a pre-moistened wipe, a moisturizing lotion, a medicated lotion, a hand sanitizer, gloves, and a disposal bag.

17. A method of carrying and accessing an undergarment and an undergarment changing accessory, said method comprising:
providing an undergarment, an undergarment changing accessory, and first and second sheet portions, said first and second sheet portions releasably sealed to one another along a first perimeter portion and along a divider to define at least two chambers in which said undergarment and said undergarment changing accessory are separately sealed, and said first and second sheet portions substantially fixed along a second perimeter portion;
grasping said first and second sheet portions at the first perimeter portion;
pulling said first and second sheet portions away from one another to open the releasably sealed first perimeter portion and the divider while leaving the first and second sheet portions fixed together along the second perimeter portion; and
removing the undergarment and the undergarment changing accessory from said first and second sheet portions.

18. The method of claim 17, further comprising:
providing a fixedly sealed portion along said second perimeter of said first and second sheet portions; and
laying the first and second sheet portions on a support surface for use as a changing pad, said first and second sheet portions remaining joined to one another at the fixedly sealed portion.

19. The method of claim 17, further comprising:
providing a folded region along said second perimeter of said first and second sheet portions, which comprise a continuous sheet;
unfolding said folded region to form an unfolded region; and
laying the first and second sheet portions on a support surface for use as a changing pad, said first and second sheet portions remaining joined to one another at the unfolded region.

20. The method of claim 17, wherein said undergarment comprises a disposable diaper.
